(19) United States
(12) Patent Application Publication
(10) Pub. No.: US 2018/0028108 A1
(43) Pub. Date: Feb. 1, 2018

(54) DIGITAL WOUND ASSESSMENT DEVICE AND METHOD

(71) Applicant: BIO1 SYSTEMS, LLC, San Carlos, CA (US)

(72) Inventors: Lauren Aquino SHLUZAS, San Carlos, CA (US); Alan SHLUZAS, San Carlos, CA (US); Gabriel ALDAZ, Palo Alto, CA (US); Joel SADLER, San Francisco, CA (US); David PICKHAM, Redwood City, CA (US); Etienne Guillaume DUMESNIL, San Carlos, CA (US); Shantanu JOSHI, Menlo Park, CA (US)

(73) Assignee: BIO1 SYSTEMS, LLC, San Carlos, CA (US)

(21) Appl. No.: 15/551,188

(22) PCT Filed: Mar. 18, 2016
(86) PCT No.: PCT/US16/23176

§ 371 Date: Aug. 15, 2017

Related U.S. Application Data
(60) Provisional application No. 62/134,580, filed on Mar. 18, 2015.

(51) Int. Cl. A61B 5/00; G06T 7/00; G06T 11/60; G06T 7/50; A61B 5/107; G06T 7/60

(52) U.S. Cl. CPC A61B 5/445; A61B 5/0077; A61B 5/1072; G06T 7/60; G06T 11/60; G06T 7/50; G06T 7/0012; A61B 90/96

(57) ABSTRACT

A wound assessment device is provided which can include any number of features. In one embodiment, the wound assessment device comprises a hands-free device that includes a camera, additional sensors, electronics including a processor, a non-transitory computer-readable storage medium, an energy source, and a display. The wound assessment device can be configured to document the healing process of a wound, and overlay a digital ruler and information pertaining to the wound and the patient onto images taken of the wound. Methods of use are also provided.

### CROSS REFERENCE TO RELATED APPLICATIONS

This application claims the benefit of U.S. Provisional Patent Application No. 62/134,580, filed Mar. 18, 2015, titled “A wearable computer with a head mounted display for hands-free image capture, measurement, speech-to-text translation, and bi-directional communication with external sensors”, the contents of which are incorporated by reference herein.

### INCORPORATION BY REFERENCE

All publications and patent applications mentioned in this specification are herein incorporated by reference to the same extent as if each individual publication or patent application was specifically and individually indicated to be incorporated by reference.

### FIELD

This disclosure relates generally to clinical wound documentation. More specifically, this disclosure relates to measurement systems, devices, and processes for documenting and recording the condition of a wound on a patient over time as healing occurs, and more particularly to systems and methods related to wound care in healthcare environments.

### BACKGROUND

Millions of patients every year are afflicted with wounds which require medical treatment to heal. These wounds can be a result of diabetic ulcers, pressure ulcers, bed sores, lacerations, dermatitis, cancer, or other wounds which take time to heal and require a clinician to monitor healing. The healing process of a wound occurs over time and the wound generally heals from the inside out.

Existing solutions to tracking the healing process may be classified as contact or non-contact. Contact techniques include disposable paper rulers, depth gages, transparency tracings, and volume measurement using silicone-based casts or saline. Non-contact techniques include photography, video image analysis, structured light and lasers, and stereophotogrammetry.

Medical professionals typically document the healing process of a wound by photographing the wound and manually entering information relating to the wound into the patient's medical chart. FIG. 1 shows this method of documenting the healing of a wound 10 by placing a ruler 20 or other measurement device next to the wound and taking a picture with a camera 30. As the healing occurs, the wound margins become closer and the wound becomes shallower. Over time, color is an important indicator of healing progression. Pink granulated tissue is evidence of wound healing, while cream, yellow, green, black or other color may be evidence of non-healing and possibly infection. This process is typically repeated until the wound is healed.

The process of taking the digital photograph of the wound and measuring the size of the wound is performed by caregivers who are typically members of the nursing or physician staff. The imaging process typically involves acquiring the patient's name, date of birth, and other identifying information from the patient or the patient's chart. This information is typically manually recorded for later physical attachment to the photograph. This information is also typically hand written on a paper ruler prior to taking wound measurements. Then the patient is positioned for acquiring the image of the wound. Depending upon where the wound is located, positioning the patient can require one or more clinicians.

The image typically includes a ruler or other measurement device, with the patient's personal identification information written on the ruler, placed next to the wound for scaling and measurement of the wound margins. These margins may be very irregular in morphology making accurate measurement difficult. The ruler is typically adhered to the patient or held in the image with one hand while the camera is operated with the other hand of the same clinician or a second clinician providing assistance.

Once the image is captured, measurements and patient data are appended to the image or the patients chart manually, which takes clinician time and has the opportunity for error. Clinical decisions are made based on the accuracy of this information. Finally, the image with all measurements, documentation, and patient data is appended to the patient's medical records, which could be an electronic medical record, or a traditional paper medical record. Extensive time is required to take the wound image, record the data, record the wound notes, enter the data, and append all the information to the patient's medical record. The delay time from wound photography to entering the patient's medical record, with the above procedure, can be up to 7 days. There is also a security concern with a potential loss of Protected Health Information (PHI) when using a process as described with multiple transfer points.

Wound assessment procedures require clinicians to accurately ‘stage’ and document a wounds classification based on current best practice guidelines. This requires the clinician to use clinical skill and judgment to adjudicate between the different classifications, based on the wounds appearance. Specific knowledge and training is required to perform this accurately, and is shown to result in considerable inter rater variability.

Another less-used technique for documenting wound healing utilizes a handheld laser scanner. A typical laser wound documentation system includes a laser scanner to measure the wound in three-dimensions. The wound can be documented on a computer system at the bedside. This system automates the measurement function of the wound documentation, but does not eliminate the need for manual patient information and wound note recording. It also does not eliminate the need for a second clinician for patient positioning, as laser scanners are typically very large and require the operator to use both hands to operate the device and the attached computer system.

### SUMMARY OF THE DISCLOSURE

A method of assessing a healing process of a wound is provided, comprising the steps of capturing a digital image of the wound with a wound assessment device, automatically determining one or more dimensions of the wound with the wound assessment device, overlaying a digital ruler onto the digital image of the wound with the wound assessment device to create a modified digital image, and displaying the digital image on a display of the wound assessment device.

In some embodiments, the automatically determining step further comprises automatically determining one or more dimensions of the wound with the wound assessment device without positioning a physical ruler or object of known size on or near the wound.

In one embodiment, the capturing step further comprises capturing the digital image of the wound with a camera of the wound assessment device.

In another embodiment, the automatically determining step further comprises determining a focal length of the camera, determining a field of view of the camera, and calculating the one or more dimensions of the wound based on the focal length of the camera and the field of view of the camera.

In one embodiment, the automatically determining step further comprises determining a distance between a range finder of the wound assessment device and the wound, determining a field of view of the camera, and calculating the one or more dimensions of the wound based on the distance and the field of view of the camera.

In some embodiments, the digital ruler conveys at least one of a height and width of the wound.

In another embodiment, the method further comprises determining patient information with the wound assessment device.

In some embodiments, the determining patient information step further comprises scanning the patient information from a bar code or quick response code positioned on or near the patient.

In some embodiments, the overlaying step further comprises overlaying the patient information and the digital ruler onto the digital image of the wound with the wound assessment device to create the modified digital image.

In one embodiment, the method further comprises repeating the capturing, automatically determining, overlaying, and displaying steps periodically throughout the healing process of the wound.

In other embodiments, the method comprises displaying multiple modified digital images simultaneously on the display of the wound assessment device. In some embodiments, the method includes adjusting a transparency of the multiple modified digital images.

In one embodiment, the capturing step further comprises capturing the digital image of the wound and of a decal positioned near the wound with the wound assessment device.

In one embodiment, the method further comprises determining patient information from the decal with the wound assessment device. The patient information can include a patient name, a wound type, or a patient identification number.

In another embodiment, the method further comprises determining a curvature of the wound from the decal with the wound assessment device.

In some embodiments, the method further comprises determining three-dimensional characteristics of the wound from the decal with the wound assessment device.

In other embodiments, the method further comprises adjusting a white balance of the digital image based on the decal with the wound assessment device.

A wound assessment device is provided, comprising a frame adapted to be worn on a head of a user, a camera disposed on or in the frame and configured to capture a digital image of a wound of a patient, a display disposed on or in the frame and configured to display the digital image to the user, a processor disposed on or in the frame and configured to control operation of the camera and the display, a non-transitory computer-readable storage medium disposed on or in the frame and configured to store a set of instructions executable by the processor, and an energy source disposed on or in the frame and configured to provide power to the camera, the display, the processor, and the non-transitory computer-readable storage medium, wherein the processor is configured to automatically determine one or more dimensions of the wound based on the digital image, the processor also being configured to overlay a digital ruler onto the digital image of the wound to create a modified digital image, the processor being further configured to present the modified digital image to the display so as to display the modified digital image to the user.

In one embodiment, the processor is configured to automatically determine one or more dimensions of the wound without positioning a physical ruler or object of known size on or near the wound.

In another embodiment, the processor is configured to automatically determine one or more dimensions of the wound by determining a focal length of the camera, determining a field of view of the camera, and calculating the one or more dimensions of the wound based on the focal length of the camera and the field of view of the camera.

In some embodiments, the device further comprises a range finder disposed on or in the frame.

In some embodiments, the processor is configured to automatically determine one or more dimensions of the wound by determining a distance between the range finder and the wound, determining a field of view of the camera, and calculating the one or more dimensions of the wound based on the distance and the field of view of the camera.

In one embodiment, the digital ruler conveys at least one of a height and width of the wound.

In another embodiment, the processor is also configured to overlay patient information onto the digital image of the wound.

In additional embodiments, the processor is configured to present a plurality of modified digital images to the display for simultaneous display of the digital images to the user.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 illustrates a conventional approach to wound assessment and documentation in which a clinician takes a photograph of a wound with a physical ruler applied to the patient in the field of view of the camera.

FIGS. 2A-2B illustrate one embodiment of a wound assessment device including its associated electronics.

FIGS. 3A-3B illustrate various embodiments for automatically determining the dimensions of a wound with a wound assessment device.

FIGS. 4A-4B illustrate one embodiment of an image captured by the wound assessment device and including a digital ruler overlaid on the image.

FIGS. 5A-5E illustrate various embodiments of decals that can be positioned in proximity to a wound being documented to aid in the assessment and documentation of that wound.

FIGS. 6A-6C illustrate one embodiment of a wound documentation system and process with a wound assessment device.

FIG. 7 illustrates a flowchart describing methods of use of the wound assessment device.

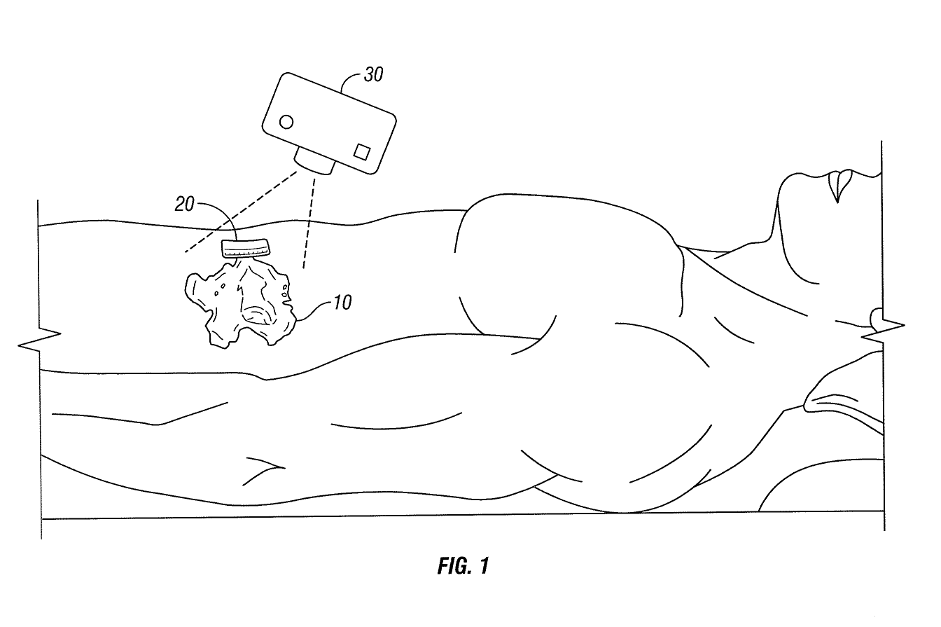

### DETAILED DESCRIPTION

This disclosure provides wound assessment devices and methods for measuring and documenting the healing process of wounds such as diabetic ulcers, pressure ulcers, bed sores, lacerations, dermatitis, cancer, or other wounds which take time to heal and require a clinician to monitor healing.

The wound assessment device described herein can be a hands-free device wearable by the clinician that can include a camera and electronics including a processor, a non-transitory computer-readable storage medium storing a set of instructions capable of being executed by the processor, a display, and an energy source such as a battery to power the device. In some embodiments, the device can also include additional sensors, such as infra-red sensor, optical sensor, ultrasound sensor, acoustic sensor, a laser, a thermal sensor, or the like. The camera and electronics can be mounted on or in a frame such as glasses frames to be worn on the head of the clinician as glasses.

This disclosure provides a hands-free wound assessment device that reduces the time and number of clinicians required for wound documentation. This device can also include the capability of automatically recording patient data from charts or wristbands and automatically appending wound assessment and patient data to a digital image of the wound before uploading the information to the patient's electronic medical record.

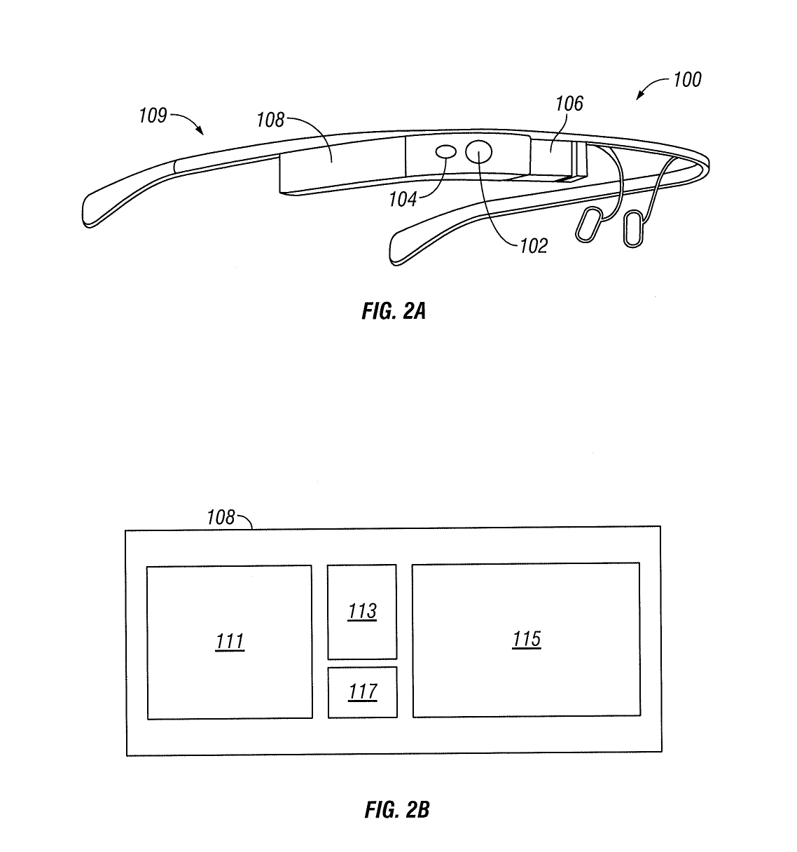

FIGS. 2A-2B illustrate one embodiment of a wound assessment device 100, that can include a camera 102, additional sensor(s) 104, display 106, electronics compartment 108, and frame 109. In this embodiment, the frame comprises glasses frames and can be worn on the head of a clinician supported by the clinician's ears and nose. As shown in FIG. 2B, the electronics compartment can house a processor 111, a non-transitory computer-readable storage medium 113 configured to store a set of instructions capable of being executed by the processor, and an energy source 115 such as a battery to power the device. The electronics compartment can also include additional electronics 117 which can be a microphone, wireless communications electronics such as WiFi, cellular, or Bluetooth chips that enable the wound assessment device to communicate with other devices and computers wirelessly; imaging processing microchips, gyroscopic position and orientation sensors, eye tracking sensors, eye blink sensors, touch sensitive sensors, speakers, vibratory haptic feedback transducers, stereoscopic cameras, or other similar electronics and hardware typically found on smartphones and digital devices. While the wound assessment device 100 is illustrated as a hands-free, wearable device, in other embodiments the wound assessment device can be a smartphone, PC, tablet, or other electronic device that includes the components described above including a camera, a processor, non-transitory computer-readable storage medium, a display, and an energy source.

The processor 111 can be configured to control the operation of the wound assessment device, including executing instructions and/or computer code stored on the non-transitory computer-readable storage medium 113, processing data captured by the camera 102 and additional sensor(s) 104, and presenting information to the display 106 for display to the user of the device. In some embodiments, the processor is configured to determine the dimensions of the wound and to overlay a digital ruler or measurement scale on top of digital images of the wound for documentation purposes. In some embodiments, the processor can determine the dimensions of the wound without requiring a physical measurement device or reference marker to be positioned on or near the wound. The modified image with the overlaid digital ruler or measurement scale can be stored on the non-transitory computer-readable storage medium 113, displayed on the display 106, stored in the patient's electronic medical record, and/or transmitted to another computer or device for storage, display, or further manipulation or study.

The processor can further be configured to affix or overlay patient information such as name, date of birth, and other identifying information from the patient or the patient's chart onto digital images of the wound. This information can be acquired automatically by the processor from the digital images, can be entered manually by the clinician, can be scanned from the patient's record and/or from a barcode wristband of the patient, or can be verbally spoken into the microphone of the wound assessment device and processed with speech recognition software. Additionally, the processor 111 may be configured to offload processor intensive operations to an additional computer, mobile phone, or tablet via the wireless connections such as WiFi, cellular, or Bluetooth.

The camera 102 can be configured to capture digital images and/or high-resolution video which can be processed by the processor 111 and stored by the non-transitory computer readable storage medium 113, or alternatively, can be transmitted to a separate device for storage. The camera can include a zoom lens or a fixed focal length lens, and can include adjustable or auto-focus capabilities or have a fixed focus. In some embodiments, the images from the camera can be used to compute a distance between the device and the wound to be used to determine the dimensions of the wound. In some embodiments, the camera can be controlled to take images/video by pressing a button, either on the wound assessment device itself or on a separate device (such as a smartphone, PC, or tablet). In other embodiments, the user can use voice control to take images/video by speaking into the microphone of the wound assessment device, which can process the command with speech recognition software to activate the camera. In one embodiment, the camera 102 may be a stereoscopic camera with more than one lens which can take simultaneous images of the wound at a known camera angle between the cameras focusing on the same point of the image. The stereoscopic images along with the camera angle can be used to create a three dimensional image of the wound which can then be measured with the methods below for width, length, and depth.

The additional sensor(s) 104 can include an infra-red sensor, optical sensor, ultrasound sensor, acoustic sensor, a laser, a thermal sensor, gyroscopic position and orientation sensors, eye tracking sensors, eye blink sensors, touch sensitive sensors, speakers, vibratory haptic feedback transducers, stereoscopic cameras, or the like. The additional sensor(s) can be used to provide additional information to the processor for processing image data from the camera. For example, the additional sensor can be used to determine a distance from the wound assessment device to the wound to be used to determine the dimensions of the wound. Alternatively, the additional sensor(s) can be used to determine characteristics of the wound such as color, temperature, or depth of the wound at various locations. In one specific embodiment, the wound assessment device can determine healing margins based on wound color and/or wound temperature. For example, the wound assessment device can include a thermal sensor and/or an infra-red sensor as the additional sensors, and can be configured to take an infra-red picture of the wound to determine isotherm boundaries of the wound indicating regions of inflammation. The dimensions of the boundaries can be calculated using the same techniques as the size of the wound is calculated, and the infra-red picture can be stored or displayed with a digital ruler overlaid on the image indicating the size of the regions of inflammation. Further details on the additional sensor(s) will be provided below.

The display 106 can be a see-through display that allows a user to see through the display but also view what is being shown on the display by the wound assessment device. The display can be, for example, an OLED screen with multiple layers of glass or transparent material surrounding the OLED. While the wound assessment device 100 of FIG. 2A includes a single display 106 in front of only one eye of the user, it should be understood that in other embodiments, the wound assessment device can include two displays (one in front of each eye of the user) or a single large display that extends across the periphery of both eyes of the user.

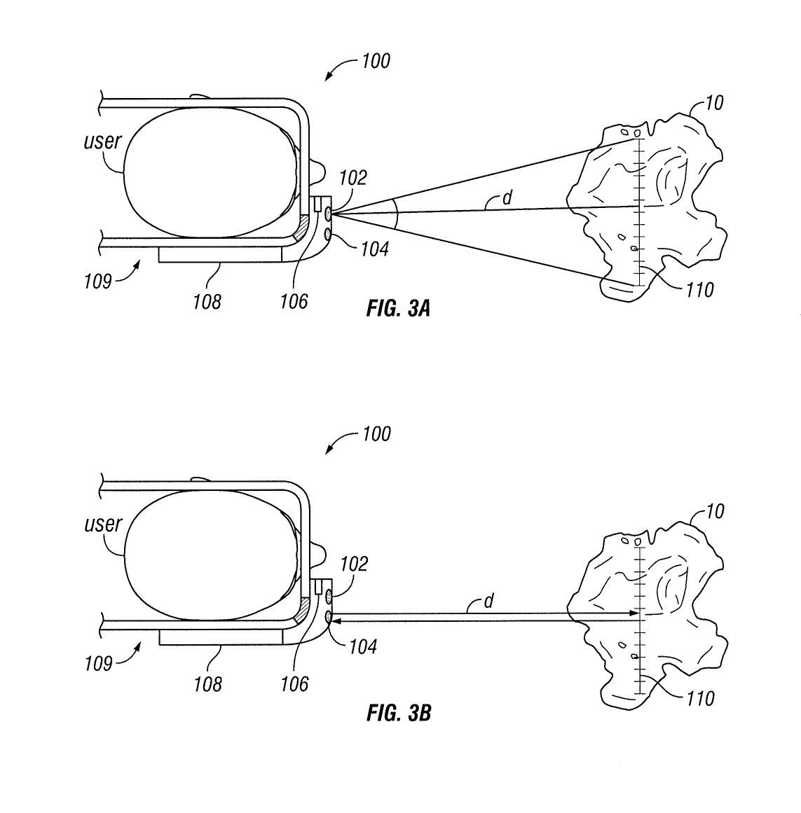

FIGS. 3A-B show top-down views of a clinician or user wearing a wound assessment device 100 as described above, including camera 102, other sensor(s) 104, display 106, electronics compartment 108, and frame 109. As described above, the processor of the wound assessment device can be configured to overlay a digital ruler or measurement scale onto digital images of the wound taken with the wound assessment device. The clinician or user can preview an image of the wound on the display of the wound assessment device with the digital ruler overlaid on top of the preview image in the display. In one embodiment, the scale of the digital image relative to the wound must be computed to create a digital ruler for placement on the digital image. One approach to compute the scale of the digital image is to determine the distance d between the wound assessment device and the wound. This distance d can be computed with the camera 102 of the wound assessment device alone, or alternatively, can be computed with additional sensor(s) 104 of the wound assessment device.

Referring to FIG. 3A, the camera 102 can have a fixed focus or an adjustable (manual or auto) focus lens. In the case of the fixed focus lens, the camera has a fixed focal length which is the distance d from the camera 102 to the wound 10 when the wound is in focus. In this embodiment, the clinician moves the wound assessment device, and thus the camera, to the proper distance from the wound to achieve an image where the wound is in sharp focus. In the case of an auto-focus lens, the lens automatically adjusts the focus of the image. The movement of the lens is used to calculate the distance d from the camera to the patient. In either approach, the distance d when the image is in focus is used in conjunction with the field of view (FOV) of the lens of the camera to scale the wound relative to the size of the image and calculate the dimensions of the wound such as the length and height of the wound. The calculated dimensions of the wound can then be overlaid on digital images of the wound as a digital ruler 110.

In one embodiment, the camera of the wound assessment device can be used to determine the depth of a wound. In this embodiment, the camera can be made to focus twice, once on the periphery of the wound and once at the center of the wound. Subtracting the difference in focal lengths d yields the wound depth at the point of focus. This could be accomplished once, or multiple times to achieve an average depth or to determine the depth of the wound at various points.

Referring to FIG. 3B, an alternate embodiment for determining the focal length d involves incorporating a range finder as the additional sensor(s) 104 to measure the distance from the wound assessment device to the wound 10. When the additional sensor(s) comprises a range finder, such as an infra-red sensor, optical sensor, ultrasound sensor, acoustic sensor, a laser, a thermal sensor, or the like, the range finder can be configured to emit energy to be reflected off of the wound and received back at the range finder. Once the distance d is computed by the additional sensor(s) 104 and the processor of the wound assessment device, the distance can be used in conjunction with the FOV of the camera to scale the wound relative to the size of the image and calculate the dimensions of the wound. The calculated dimensions of the wound can then be overlaid on digital images of the wound as a digital ruler 110.

Similar to above, the range finder can be used to determine the depth of the wound. The range finder can be used to map the wound by pointing the range finder at a point of interest in the wound, or scanning the range finder across the wound in a side to side manner. The range finder can operate continually as the wound is scanned.

In both embodiments described above, the distance between the wound assessment device, and also the dimensions of the wound, can be determined by the processor of the wound assessment device without requiring a physical scale, ruler, or other object of known size to be placed on or near the wound being imaged.

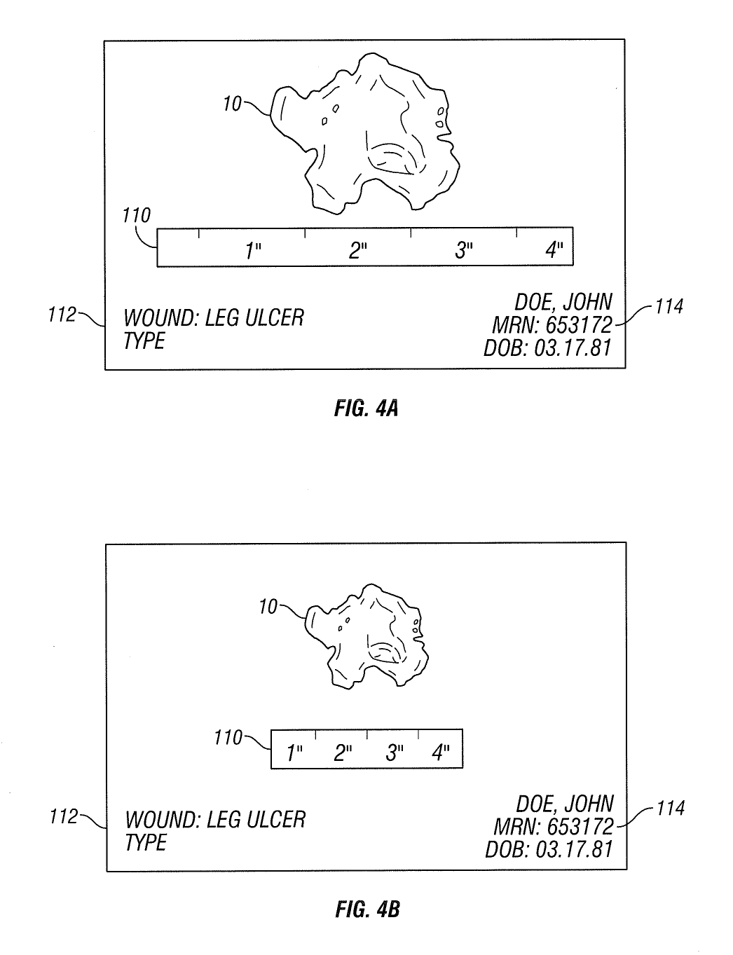

FIGS. 4A-4B illustrate one embodiment of a digital image 112 captured and processed by the wound assessment device disclosed herein. The digital image 112 can include the entirety of wound 10, and can additionally be overlaid with a digital ruler 110 and patient information 114 on the digital image. The digital ruler 110 can include at least one axis of measurement, and preferably two axes of measurement. The patient information 114 can comprise the patient's name or identifying number, the date of birth (DOB), the type of wound, and any other information that may be vital to identify the patient or care for the wound. As described above, the wound assessment device can be configured to capture a digital image of the wound and process the image to overlay a digital ruler and/or patient information on top of the digital image. The images shown in FIGS. 4A-4B can be displayed on the display of the wound assessment device, or stored on non-transitory computer readable storage medium for later review.

In FIGS. 4A-4B, two different images of the same wound are shown with different levels of magnification applied to the image. For example, the digital image 112 shown in FIG. 4A is displayed at a 100% magnification, while the digital image 112 shown in FIG. 4B is shown at 50% magnification. The size of the digital ruler 110 can be scaled automatically by the processor of the wound assessment device to accurately measure the wound even with the variable zoom of the image. Additionally, the scale of the graduations of the ruler can be varied with the level of magnification, to maintain accuracy. For example, zooming way in on an image may require the digital ruler to show measurements on the order of 1/32^{th}'s of an inch, or on the order of millimeters, while zooming out on an image may require measurements only by the inch or by the centimeter.

The wound can be measured for length, width, and/or depth by the wound assessment device using the digital ruler overlaid onto the image captured by the camera. The cross sectional area and or volume of the wound can be computed by the wound assessment device. The wound cross section can be computed by approximating the wound as an ellipse and identifying the ends of the long axis of the wound and the ends of the short axis of the wound. Wound end identification can be performed automatically via the processor of the wound assessment device, or dictated into the computer by the user.

The wound assessment device can be used to document the healing process of the wound over time. As such, several images of the wound including the digital ruler will be created during the healing process. In one embodiment, the wound assessment device can overlay multiple images taken at different times on top of each other to assess healing at or near the bedside of the patient. For example, the multiple images can be overlaid and displayed to the clinician in the display of the wound assessment device, or on the display of a smartphone, PC, or tablet. In some embodiments, the transparency of each of the individual images can be adjusted so that the clinician can view all the overlaid images simultaneously to assess the healing progress. Furthermore, the images can be scaled by the wound assessment device to allow for easy comparison. Edge detection algorithms executed by the wound assessment device can be used to trace the periphery of the wound to achieve a more accurate or precise measurement of the wound. In another embodiment, the wound assessment device can orientate captured images and calculate border margin progression based on the wound dimension information calculated with each subsequent image, thereby providing information on healing rate (distance/time) to the clinician.

Another feature of the present disclosure is the inclusion of decals which can be removably attached to the patient (e.g., with adhesive) and captured by the camera of the wound assessment device to determine additional information about the wound or the patient. For example, the decal can include bar codes, quick response codes, or other identifying markers that can be scanned by the camera of the wound assessment device to automatically update and annotate the digital images with patient information such as name, DOB, wound type, etc. Additionally, the decals can include patterns, shapes, or colors that can be used by the wound assessment device to determine additional characteristics of the wound, such as wound depth and three-dimensional measurements, curvature of the wound, and color of the wound. When applied with adhesive, the decal can be placed anywhere on the body of the patient without the need for the clinician to hold the decal during image capture.

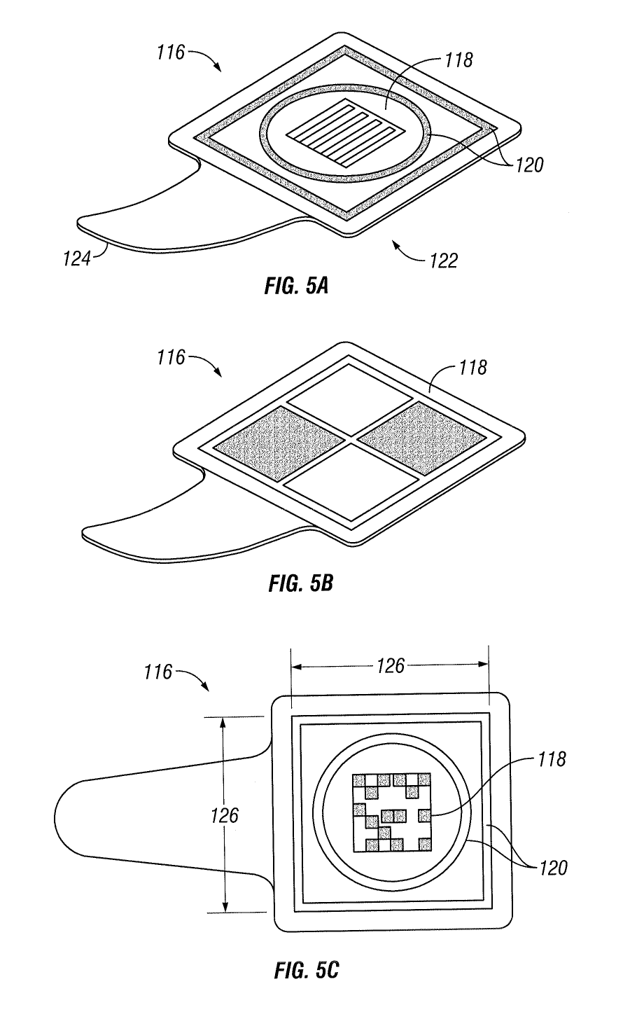

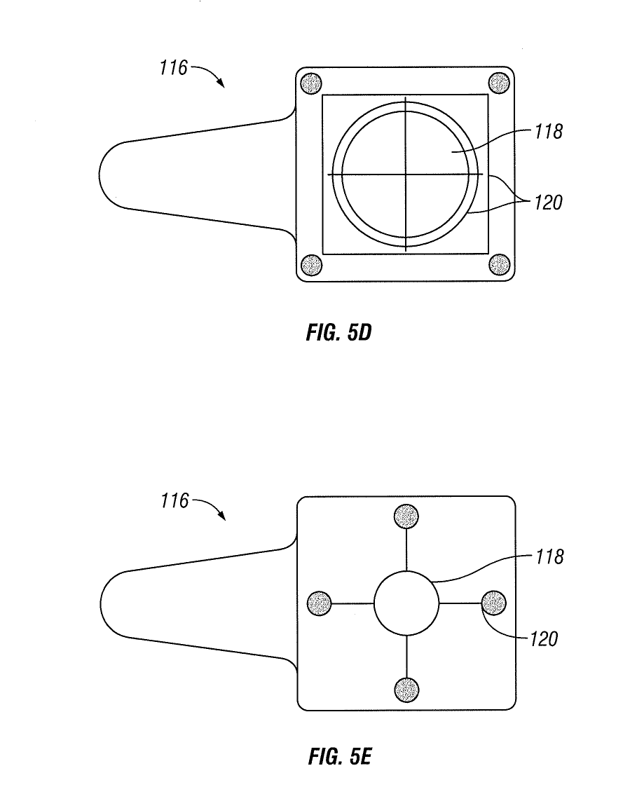

Various decals according to several embodiments are shown in FIGS. 5A-5E. FIG. 5A shows a decal 116 which includes a pattern 118 comprising a bar code and registration marks 120 comprising a circle and a square of known dimensions. The decal 116 can include an adhesive side 122 adapted to be placed on the skin or clothing of a patient, and can further include an optional tab 124 that does not include an adhesive to allow for easy removal of the decal. In some embodiments, the bar code can be read by the wound assessment device and may include patient information as mentioned above. This patient information can be automatically appended to the digital image of the wound taken by the wound assessment device.

FIG. 5B shows a decal 116 which includes a pattern 118 comprising a plurality of squares of different colors (e.g., black and white, or black and gray). The squares can be of known dimensions and can be used by the processor of the wound assessment device to determine a curvature of a wound so as to aid in determining the three-dimensional characteristics of the wound (e.g., depth). When the decal is placed on the skin of the patient, the contours of the pattern (e.g., each square) can be calculated and the three-dimensional surface of the skin may be computed by the wound assessment device. This three-dimensional surface can then be used to scale the digital ruler to the image to give an accurate representation of the actual wound size and dimensions.

When the decal is placed on a curved surface, such as near a wound, it should be understood that the decal will bend and conform to the curved surface. The pattern of the decal has a known calibration length which can be used by the wound assessment device to calibrate the image relating to the curvature of the underlying skin. When the decal bends, the length of the pattern 118 visible to the camera will be smaller or larger depending on the curvature of the decal. The wound assessment device can capture a two-dimensional image of the wound and the decal, and the pattern can be scaled relative to the size of known features of the pattern. The known length of the pattern can be compared to the captured two-dimensional length of the pattern to calculate a radius of curvature of the decal in the deformed or bent state. Since this radius of curvature corresponds to the portion of the wound near the decal, the wound assessment device can use this information to draw conclusions about the radius of curvature, and thus the depth, of the wound itself.

FIG. 5C shows another embodiment of a decal 116 which can include a pattern 118 comprising a quick response (QR) code or matrix bar code. Like the bar code illustrated in FIG. 5A, the QR code or matrix bar code can also include patient information that can be scanned by the wound assessment device. This decal can also include registration marks 120, such as a square and a circle of known dimensions. For example, the square can have a height and width of length 126, as shown. The registration marks can be used by the wound assessment device to measure the wound, calibrate the digital ruler, or determine characteristics of the wound such as the curvature of the wound or the portion of the patient's body where the wound is situated, as will be described in more detail below.

FIG. 5D shows another embodiment of a decal 116 which can include a pattern 118 comprising crossing horizontal and vertical lines of a known length, and registration features of a circle and a square. Like the plurality of squares illustrated in FIG. 5B, the crossing lines and registration marks 120 can be used to determine the measurements of the wound as well as characteristics of the wound such as curvature and depth. By capturing an image of the crossing lines and comparing the captured length of the lines to the known length of the lines, the wound assessment device can determine a radius of curvature of the decal, and thus, a radius of curvature of the wound near the location of the decal.

FIG. 5E shows yet another embodiment of a decal 116, which can include a pattern 118 comprising a pure white circle and one or more registration marks such as lines of known dimensions or circles of known diameter. In this embodiment, the pure white circle can be used to correct the white balance of images taken with the wound assessment device, and the registration marks can be used to determine measurements or characteristics of the wound. Accurate white balance is essential for proper color reproduction in the image. Other colors could be added to the decal to provide a reference for color correction due to different lighting scenarios, fluorescent lights, sunlight, led lights, amber lights, ultra violet, infra-red, xenon, and halogen.

The decals described above can be made of a metal or polymer film or paper, with a tacky adhesive on the patient contact side. In some embodiments, the decal does not include an adhesive, but instead can be placed on the patient simply by wetting the decal. When placed on the skin of the patient, the decal is configured to be identified by the wound assessment device. The decal can be smaller than a traditional adhesive ruler which allows for the decal to be placed further away from the wound, enhancing patient comfort. The decal is preferably provided sterile to minimize the chance of infection. The patterns 118 and registration features 120 of the decal can be printed, engraved, or embossed. There may be a single decal or there may be multiple decals placed around the wound to provide multiple points of registration for documenting a particularly large wound or a wound with complex contours.

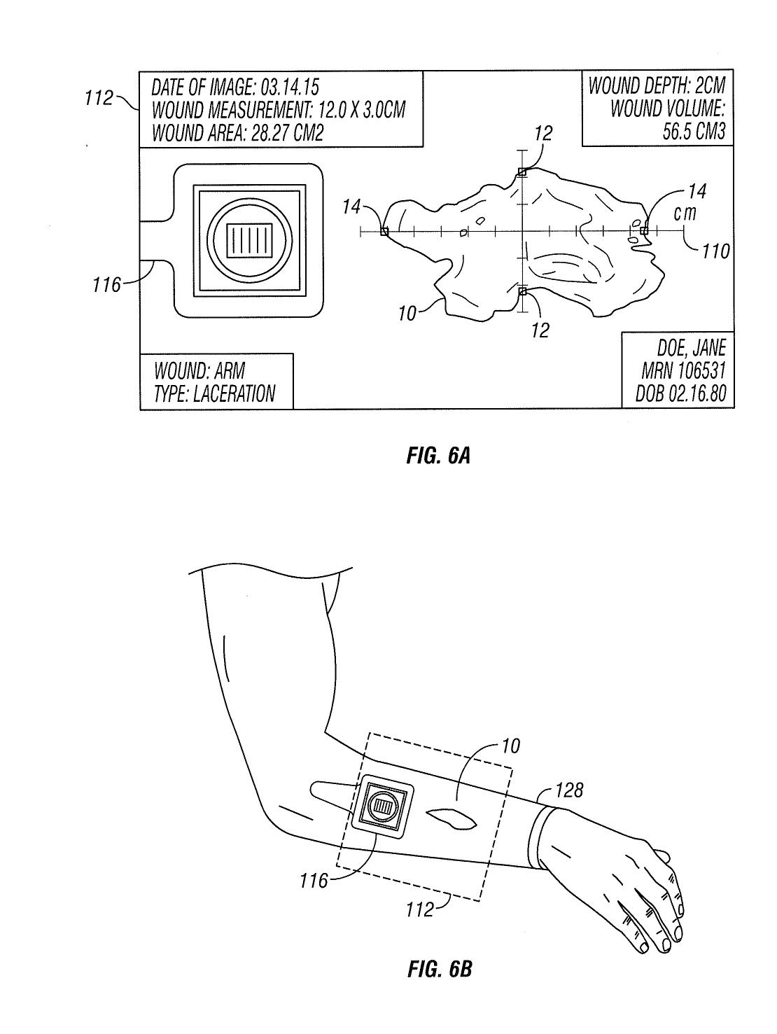

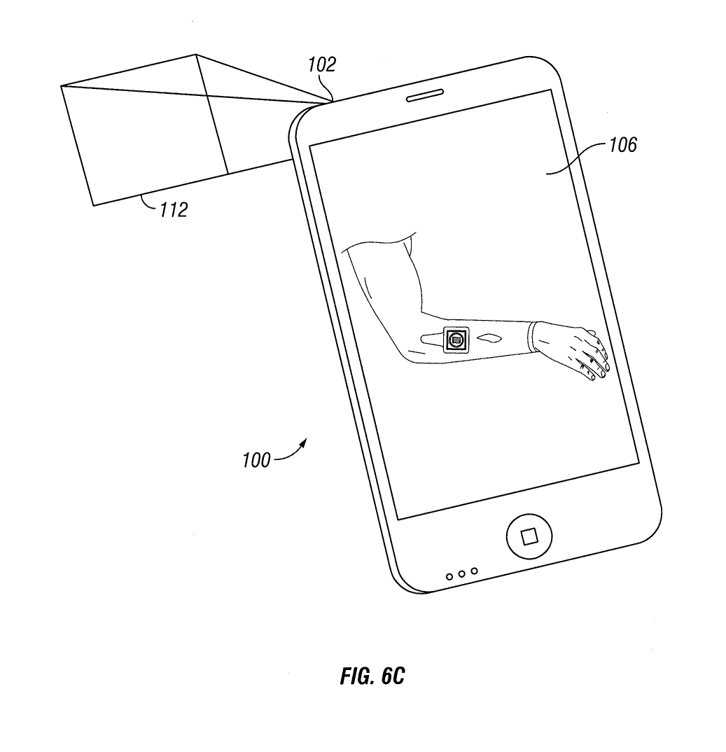

FIGS. 6A-6C illustrate the overall system and one method of use of the wound assessment device described herein. FIG. 6A shows a digital image 112 of a wound 10 with a digital ruler 110 and patient information 112 overlaid on the digital image. The digital image can also include height endpoints 12 and width endpoints 14 overlaid on the image. A decal 116, such as one of the decals described above, is also within the frame of the digital image, and can be the source of the patient information 112, for example. The height endpoints 12 and width endpoints 14 can be used by the wound assessment device to determine the area and/or volume of the wound, which can be overlaid onto the digital image and displayed to the user. The wound assessment device can also use the techniques described above to determine the depth of the wound, which can also be overlaid onto the digital image and displayed to the user. FIG. 6B is a view of the patient and the wound 10. A decal 116 is positioned near the wound, and the FOV of the digital image corresponding to FIG. 6A is shown in dotted lines over the wound 10 and decal 116. The patient can also wear a wrist band 128 or other identification that can be imaged or scanned by the wound assessment device. FIG. 6C shows a wound assessment device 100 in the form of a smartphone, including a camera 102 and display 106. The FOV of the digital image corresponding to FIG. 6A is shown in dotted lines.

Methods of use will now be described. It should be understood that any of the embodiments of the wound assessment device described above can be utilized in the methods described below.

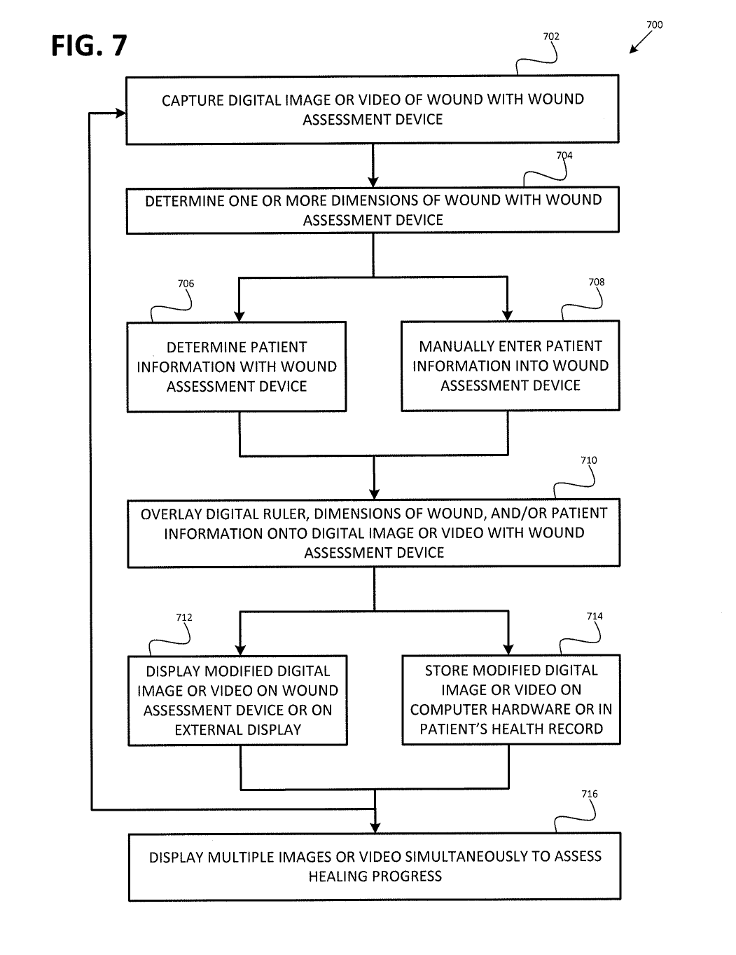

Referring to FIG. 7, a method of assessing a wound is described in flowchart 700. First, at step 702 of flowchart 700, a caregiver or clinician can capture a digital image or video of the wound to be assessed with the wound assessment device. As described above, the wound assessment device can be a hands-free device as shown and described in FIG. 2A, or can be a smartphone, PC, tablet, or other electronic device as shown and described in FIG. 6C. The caregiver or clinician should take care to capture the entire wound in the image frame of the digital image or digital video, so as to be able to determine dimensions of the entire wound.

Next, at step 704 of flowchart 700, the wound assessment device can determine one or more dimensions of the wound. For example, in one embodiment, a processor of the wound assessment device can determine the dimensions of the wound by determining a focal length from the camera of the device to the wound, and calculating the dimensions of the wound based on the focal length and the field of view of the camera. In another embodiment, the wound assessment device can utilize an additional sensor to act as a range finder to determine the distance between the additional sensor and the wound. The additional sensor can comprise, for example, an infra-red sensor, optical sensor, ultrasound sensor, acoustic sensor, a laser, a thermal sensor, or the like. In some embodiments, the dimensions of the wound can be determined by the wound assessment device alone, and does not require additional physical objects such as physical rulers or other objects of known size to be used to determine the dimensions of the wound.

Next, at steps 706 and 708 of flowchart 700, the wound assessment device can either determine patient information or the patient information can be manually entered into the wound assessment device. For example, the wound assessment device can scan a patient barcode or other physical object to automatically obtain patient information such as name, identifying number, date of birth (DOB), the type of wound, etc., or alternatively, the patient information can be entered into the device such as by dictation.

Next, at step 710 of flowchart 700, the wound assessment device can overlay a digital ruler, dimensions of the wound, and/or patient information onto the digital image or video of the wound. As described above, the digital ruler can show both the height and width of the wound. In some embodiments, the wound assessment device can calculate a volume or area of the wound and display that number on the image. The wound assessment device can also overlay the patient information onto the digital image or video of the wound.

Next, at steps 712 and 714 of flowchart 700, the modified digital image or video with the overlaid digital ruler and/or patient information can be displayed on the display of the wound assessment device, on an external display, or can be stored on computer hardware or in the patient's digital health record.

Next, steps 702-714 of flowchart 700 can be repeated periodically to assess the healing process. At step 716 of flowchart 700, the plurality of images or video acquired during multiple rounds of wound documentation can be displayed simultaneously on the display of the wound assessment device or an external display to assess the healing process. In one embodiment, the transparency of each image or video can be adjusted so as to be able to view all the images or video at once.

The data structures and code described in this detailed description are typically stored on a non-transitory computer-readable storage medium, which may be any device or medium that can store code and/or data for use by a computer system. The non-transitory computer-readable storage medium includes, but is not limited to, volatile memory, non-volatile memory, magnetic and optical storage devices such as disk drives, magnetic tape, CDs (compact discs), DVDs (digital versatile discs or digital video discs), or other media capable of storing computer-readable media now known or later developed.

The methods and processes described in the detailed description section can be embodied as code and/or data, which can be stored in a non-transitory computer-readable storage medium as described above. When a computer system reads and executes the code and/or data stored on the non-transitory computer-readable storage medium, the computer system performs the methods and processes embodied as data structures and code and stored within the computer-readable storage medium.

Furthermore, the methods and processes described above can be included in hardware modules. For example, the hardware modules can include, but are not limited to, application-specific integrated circuit (ASIC) chips, field-programmable gate arrays (FPGAs), and other programmable-logic devices now known or later developed. When the hardware modules are activated, the hardware modules perform the methods and processes included within the hardware modules.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method of assessing a healing process of a wound, comprising the steps of:
capturing a digital image of the wound with a wound assessment device;
automatically determining one or more dimensions of the wound with the wound assessment device;
overlaying a digital ruler onto the digital image of the wound with the wound assessment device to create a modified digital image; and
displaying the digital image on a display of the wound assessment device.

2. The method of claim 1, wherein the automatically determining step further comprises automatically determining one or more dimensions of the wound with the wound assessment device without positioning a physical ruler or object of known size on or near the wound.

3. The method of claim 1, wherein the capturing step further comprises capturing the digital image of the wound with a camera of the wound assessment device.

4. The method of claim 3, wherein the automatically determining step further comprises:
determining a focal length of the camera;
determining a field of view of the camera; and
calculating the one or more dimensions of the wound based on the focal length of the camera and the field of view of the camera.

5. The method of claim 1, wherein the automatically determining step further comprises:
determining a distance between a range finder of the wound assessment device and the wound;
determining a field of view of the camera; and
calculating the one or more dimensions of the wound based on the distance and the field of view of the camera.

6. The method of claim 1, wherein the digital ruler conveys at least one of a height and width of the wound.

7. The method of claim 1, further comprising determining patient information with the wound assessment device.

8. The method of claim 7, wherein the determining patient information step further comprises scanning the patient information from a bar code or quick response code positioned on or near the patient.

9. The method of claim 7, wherein the overlaying step further comprises overlaying the patient information and the digital ruler onto the digital image of the wound with the wound assessment device to create the modified digital image.

10. The method of claim 1, further comprising repeating the capturing, automatically determining, overlaying, and displaying steps periodically throughout the healing process of the wound.

11. The method of claim 10, further comprising displaying multiple modified digital images simultaneously on the display of the wound assessment device.

12. The method of claim 11, comprising adjusting a transparency of the multiple modified digital images.

13. The method of claim 1, wherein the capturing step further comprises capturing the digital image of the wound and of a decal positioned near the wound with the wound assessment device.

14. The method of claim 13, further comprising determining patient information from the decal with the wound assessment device.

15. The method of claim 14, wherein the patient information can include a patient name, a wound type, or a patient identification number.

16. The method of claim 13, further comprising determining a curvature of the wound from the decal with the wound assessment device.

17. The method of claim 13, further comprising determining three-dimensional characteristics of the wound from the decal with the wound assessment device.

18. The method of claim 13, further comprising adjusting a white balance of the digital image based on the decal with the wound assessment device.

19. A wound assessment device, comprising:
a frame adapted to be worn on a head of a user;
a camera disposed on or in the frame and configured to capture a digital image of a wound of a patient;
a display disposed on or in the frame and configured to display the digital image to the user;
a processor disposed on or in the frame and configured to control operation of the camera and the display;
a non-transitory computer-readable storage medium disposed on or in the frame and configured to store a set of instructions executable by the processor; and
an energy source disposed on or in the frame and configured to provide power to the camera, the display, the processor, and the non-transitory computer-readable storage medium;
wherein the processor is configured to automatically determine one or more dimensions of the wound based on the digital image, the processor also being configured to overlay a digital ruler onto the digital image of the wound to create a modified digital image, the processor being further configured to present the modified digital image to the display so as to display the modified digital image to the user.

20. The device of claim 19, wherein the processor is configured to automatically determine one or more dimensions of the wound without positioning a physical ruler or object of known size on or near the wound.

21. The device of claim 19, wherein the processor is configured to automatically determine one or more dimensions of the wound by determining a focal length of the camera, determining a field of view of the camera, and calculating the one or more dimensions of the wound based on the focal length of the camera and the field of view of the camera.

22. The device of claim 19, further comprising a range finder disposed on or in the frame.

23. The device of claim 22, wherein the processor is configured to automatically determine one or more dimensions of the wound by determining a distance between the range finder and the wound, determining a field of view of the camera, and calculating the one or more dimensions of the wound based on the distance and the field of view of the camera.

24. The device of claim 19, wherein the digital ruler conveys at least one of a height and width of the wound.

25. The device of claim 19, wherein the processor is also configured to overlay patient information onto the digital image of the wound.

26. The device of claim 19, wherein the processor is configured to present a plurality of modified digital images to the display for simultaneous display of the digital images to the user.
